(19) United States
(12) Patent Application Publication
(10) Pub. No.: US 2022/0175693 A1
(43) Pub. Date: Jun. 9, 2022

(54) DIETARY SUPPLEMENT COMPRISING LUTEIN FOR TELOMERE PROTECTION, AND METHOD FOR PRODUCTION THEREOF

(71) Applicant: BIOGEN EXTRACTS PRIVATE LIMITED, Bangalore (IN)

(72) Inventor: Jai Shankar Raghava Chandra, Bangalore (IN)

(73) Assignee: BIOGEN EXTRACTS PRIVATE LIMITED, Bangalore (IN)

(21) Appl. No.: 17/603,348

(22) PCT Filed: Apr. 15, 2020
(86) PCT No.: PCT/IB2020/053553

§ 371 Date: Oct. 13, 2021

(30) Foreign Application Priority Data
Apr. 16, 2019 (IN) 201941015231

(51) Int. Cl. A61K 31/047; A61K 31/355; A61K 36/28; A61K 45/06; A61P 39/06

(52) U.S. Cl. CPC A61K 31/047; A61K 31/355; A61K 36/28; A61K 45/06; A61K 2236/37; A61K 2236/31; A61K 2236/53; A61K 2236/51; A61K 2236/333; A61P 39/06

(57) ABSTRACT

This invention discloses a dietary supplement composition. More particularly, the invention deals with the method of production of dietary supplement composition comprising natural lutein and tocopherols, optionally along with acceptable additives to a subject, wherein the dietary supplement composition reduces, ameliorates, prevents, or reverses the oxidative DNA damage and slows telomere shortening. Invention further describes a method for promoting detoxification in cells of a subject, including administering to the subject an oral formulation comprising a plurality of agents that stimulate natural stem cell production within the body; repair the stem cells fibroblasts; pro-long the replicative lifespan; slow the telomere shortening; improve the telomerase activation in stem cells; improve the telomerase activation in body cells; promote the health-span in humans; and reduce the oxidative stress. The present invention can be used for the development of functional food, dietary plan and as a nutritional supplement.

### PRIORITY PARAGRAPH

This application claims priority to the Indian provisional patent application No. 201941015231, filed on Apr. 16, 2019, titled “DIETARY SUPPLEMENT COMPRISING LUTEIN FOR TELOMERE PROTECTION, AND METHOD FOR PRODUCTION THEREOF” and is incorporated herein by reference.

### TECHNICAL FIELD OF THE INVENTION

This invention belongs to the field of dietary supplement composition. More particularly, the invention deals with the method of production of dietary supplement composition comprising natural lutein and tocopherols, optionally along with acceptable additives to a subject, wherein the dietary supplement composition reduces, ameliorates, prevents, or reverses the oxidative DNA damage and slows telomere shortening.

### BACKGROUND OF THE INVENTION

Several efforts have been made to develop formulations and treatments to slow the aging process in humans. First, it is important to understand the cause, to develop a remedy.

Current theories suggest that aging is a complex biological process and many factors are interlinked that affect aging process. One of the well-established theories of aging is DNA and genetic theories.

DNA is the blueprint of life and a type of genetic clock. DNA is easily oxidized and this damage can be due to diet, toxins, pollution, radiation, lifestyle and so on. Thus, each individual has the ability to accelerate DNA damage or slow it down. One recent theory of DNA damage is the telomerase theory of aging.

Telomeres are long stretches of repeated sequences that cap the ends of chromosomes and are believed to stabilize the chromosome. In humans, telomeres are typically 7-10 kb in length and comprise multiple repeats of the sequence -TTAGGG.

A telomere shortens with each cell division and progressively limits the replicative potential of normal human somatic cells. A clear mechanism had been identified for a biological clock that could measure mitotic time and account for the phenomenon of cell replicative senescence. The timing of senescence depends primarily on the replicative history of the cells (the number of elapsed cell divisions) and much less on the passage of chronological time.

This shortening of telomeres lead to cellular damage as the cell cannot duplicate correctly. It is believed that each time a cell divides it duplicates itself a little less accurately than the previous time. This eventually leads to cellular dysfunction, aging and death.

Telomerase is a ribonucleoprotein that catalyzes the addition of telomeric repeats to the ends of telomeres.

Telomerase is not expressed in most adult cells, and telomere length decreases with successive rounds of replication. After a certain number of rounds of replication, the progressive shortening of the telomeres results in the cells entering a telomeric crisis stage, which in turn leads to cellular senescence. Certain diseases are associated with rapid telomeric loss, resulting in premature cell senescence. Expression of the gene encoding the human telomerase protein in human cells has been shown to confer an immortal phenotype, presumably through bypassing the cells' natural senescence pathway. In addition, expression of the telomerase gene in aging cells with short telomeres has been shown to produce an increase in telomere length and restore a phenotype typically associated with younger cells.

In relation to the above hypothesis, one of the important factors is oxidative stress that accelerates telomere loss, whereas antioxidants decelerate it. Hence, oxidative stress is an important modulator of telomere loss and that telomere-driven replicative senescence is primarily a stress response. This might have evolved to block the growth of cells that have been exposed to a high risk of mutation.

So, there is a need in the art to develop a dietary food supplement with anti-oxidative properties and healthy aging choices, having direct effect on telomerase that will prolong the life span of cells.

In addition, such food supplement can slow down the telomere shortening and pro-longing the replicative lifespan of cells.

There are different kinds of antioxidants are available, such as quercetin, hesperidin, alpha lipoic acid (ALA), N-acetyl-L-cysteine (NAC), lutein, lycopene, astaxanthin, plant sterols, isoflavones, garlic extract, green tea extract, cruciferous vegetables (and their extracts), fruit blends, coenzyme Q-10, and resveratrol to name a few.

In nature, lutein abundantly occurs as mono and di-esters of long chain fatty acids like palmitic, stearic, myristic, oleic, linoleic, lauric and pentadecanoic acids. The lutein is assimilated by the body in the natural ester form are biologically hydrolyzed inside the human body.

### PRIOR ART DOCUMENTS

PATENT 20140023701 DISCLOSED A METHOD AND COMPOSITION FOR AMELIORATING THE EFFECTS TOR A SUBJECT EXPOSED TO RADIATION OR OTHER SOURCES OF OXIDATIVE STRESS

Antioxidant and chemopreventative agent mixtures contain non-essential natural antioxidants and chemopreventative agents, including rutin, quercetin, hesperidin, alpha lipoic acid (ALA), N-acetyl-L-cysteine (NAC), lutein, lycopene, astaxanthin, plant sterols, isoflavones, garlic extract, green tea extract, cruciferous vegetables (and their extracts), fruit blends, coenzyme Q-10, and resveratrol. It may also contain other similar non-essential antioxidant ingredients that are botanical, nutritional, dietary, additive, or otherwise. Generally Recognized as Safe (GRAS) and allowed within the Carotenoids such as lutein and zeaxanthin appear to be more efficiently absorbed when administered with high-fat meals. They are hydrolyzed in the small intestine via esterates and lipases, and solubilized in the lipid core of micelles formed from bile acids and other lipids. They can also form clathrate complexes with conjugated bile salts.

PATENT U.S. Pat. No. 7,368,481B1 DISCLOSED A PET ANTI-AGING WELLNESS SUPPLEMENT FOR CATS which is a health and nutrition supplement dosage for pets, particularly feline pets, consisting essentially of anti-oxidant vitamins, B complex vitamins, bioflavonoids, chelated minerals, digestive enzymes, herbs, nutrients, and essential fatty acids and amino acids. Incidentally it contained lutein 1.0 mcg from marigold as one of the anti-oxidant entity and Vitamin E (d-Alpha Tocopheryl acetate) 5.0 IU in association with many other conventional nutritional constituents. But nothing has been revealed about the effects of lutein on telomerase of human fibroblast cells.

PATENT US20040001817A1 disclosed an anti-aging nutritional supplement. An anti-aging nutritional supplement composition includes vitamins, minerals, an inflammatory process support, a blood sugar/insulin support, a botanical antioxidants, a methylating factor, a DNA repair agent, a fat metabolizer, an absorption enhancer, a brain function support, whole foods, a cellular energizer, a nucleotide precursor, amino acids, a fatty acid complex, and digestive enzymes. The composition supplies nutritional supplements necessary for proper glycation, DNA methylation, anti-oxidation, and control of inflammatory processes. The composition and the method of use provide an effective anti-aging treatment by decreasing DNA damage, increasing DNA repair, and improving immune function of human body that contained botanical antioxidants, including a blend of 147 mg of cruciferous vegetable concentrate: broccoli, kale, radish (2% glucosinolates), grape skin extract (37% total polyphenols), tomato lycopene extract (20% lycopene), rosemary 4:1 extract (aerial parts), pycnogenol (pine tree bark extract) and lutein (from marigold flower extract). But specifically, lutein action on telomerase is not disclosed.

PATENT 8974839 disclosed a dietary supplement system includes a dietary supplement composition for oral administration by an individual in the morning, the composition, including (a) a telomere maintenance complex including: Purslane extract (aerial parts); Turmeric rhizome extract (95% curcuminoids); Quercetin dehydrate, Cayenne pepper fruit; Vanadium (as vanadyl sulfate); Fenugreek seed; Astragalus root extract, Omega fatty acid complex including linoleic acid; alpha-linolenic acid; oleic acid borage seed oil gamma-linolenic acid), evening primrose oil fish body oil (eicosapentaenoic acid; docosahexaenoic acid); (b) a calorie restriction mimetics and gene expression complex including Trans-resveratrol (from Polygonum cuspidatum root extract); Pterostilbene Fisetin 50% (Buxus microphlla Sieb (stem and leaf; Alpha lipoic acid, Coenzyme Q-10, Betaine HCl, Sulfur (from methylsulfonylmethane); L-Carnitine tartrate; L-Carnitine HCl, and (c) a free radical scavenger complex, including Green tea leaf extract catechin and polyphenols); Anthocyanins (from bilberry fruit and grape skin extracts).

A dietary supplement system includes a first dietary supplement composition for oral administration by an individual in the morning, comprising: (a) a telomere maintenance complex comprising: Purslane extract (aerial parts), 4-6 mg; Turmeric rhizome extract (95% curcuminoids), 20-30 mg; Quercetin dehydrate, 12-18 mg; Cayenne pepper fruit, 8-12 mg; Vanadium (as vanadyl sulfate), 20-30 meg; Fenugreek seed, 20-30 mg; Astragalus root extract, 8-12 mg; Omega fatty acid complex, 160-240 mg, including linoleic acid (10.6%), alpha-linolenic acid (1.3%), oleic acid (1.6%), borage seed oil (10% gamma-linolenic acid), evening primrose oil (4.8% GLA), fish body oil (4.5% eicosapentaenoic acid, 3.0% docosahexaenoic acid); (b) a calorie restriction mimetics and gene expression complex comprising: Trans-resveratrol (from Polygonum cuspidatum root extract), 1.6-2.4 mg Pterostilbene (99%), 1.6-2.4 mg; Fisetin 50% (Buxus microphlla Sieb (stem and leaf)), 1.6-2.4 mg; Alpha lipoic acid, 8-12 mg; Coenzyme Q-10, 4-6 mg; Betaine HCl, 3.2-4.8 mg; Sulfur (from methylsulfonylmethane), 1-1.5 mg; L-Carnitine tartrate, 8-12 mg; L-Carnitine HCl, 4-6 mg; and (c) a free radical scavenger complex, comprising: Green tea leaf extract (40% catechin and polyphenols), 40-60 mg; Anthocyanins (from bilberry fruit and grape skin extracts), 4-6 mg. Claims of 8974839 (c) a free radical scavenger complex comprising: Cruciferous vegetable concentrate; Grape skin extract; Tomato lycopene extract; Rosemary extract; Pycnogenol; Lutein; and Green barley grass.

PATENT 8974839 US disclosed a dietary supplement for oral consumption containing telomerase stabilizing complex, it contained a number of various natural plant extracts, inorganic constituents, along with free radical scavenger complex containing lutein. But it did not disclose the lutein extraction for marigold and exclusive anti-ageing effect by the damage controlling of telomerase of stem cell fibroblasts as revealed by present invention.

PATENT EP2008661A2 disclosed a formulation based on marigold aloe and centellae. The formulation includes between 1 and 98 parts of glycolic extract of marigold officinalis, between 1 and 98 parts of glycolic extract of aloevera and between 1 and 98 parts of glycolic extract of Centellae asiaticae.

The formulation conforms an emulsion from the use of between 4 and 8% of glycolic extract of marigold officinalis, between 4 and 8% of glycolic extract of aloe vera and between 5.5 and 9% of glycolic extract of Centellae asiaticae incorporated into a base cream nonionic that includes an oily phase where it is included between 6.5 and 12% of self emulsive wax nonionic; between 3.2 and 5,8% of estearic acid of triple pressure; between 4 and 6,4% of solid Vaseline (petroleum jelly) and between 7.6 and 9,7% of liquid Vaseline (petroleum jelly).

In turn, the watery phase includes between 7 and 10,4% of glycerine; between 0.1 and 0,8% of imidozolindil urea; between a 1.6 and 3,3% of preserving universal; between 0.7 and 1,5% of polyethilenglycol 400; between 0.35 and 0,78% of Twen 80. Finally, the emulsion includes the addition of demineralized water up to completing 100 g of product.

WO2013066623A1 disclosed anti-aging application and method for treating aging. There is a topical anti-aging composition which is configured to interfere with the production of tyrosinase, as well as melanin by reacting with melanocytes as well as reacting with, or acting on keratinocytes. The anti-aging composition comprises an emulsion comprising a plurality of active ingredients comprising a plurality of pigmentation reducing elements which are configured to interrupt or prevent the production of tyrosinase and melanin. In addition, there are a plurality of antioxidants a plurality of anti-wrinkling agents a plurality of anti-inflammatory agents at least one additional agent comprising at least one of an emollient, a proactant and a conditioning agent. Incidentally contained extract of Calendula.

The present invention of dietary supplements comprising anti-oxidant and healthy-aging properties describes the extraction marigold flower buds by saponification, neutralization and recrystallization, stabilization with oil based tocopherol (an antioxidant) and application of lutein crystals along with tocopherol is novel. The novelty aspect of Lute-gen lies in the method of administering the lutein+tocopherol composition for reducing the shortening rate of telomerase of fibroblast cells and increasing the proliferation rate of telomerase and thereby increasing the health span of humans by way of dietary supplementation.

Therefore, the said invention overcomes all the limitations of available prior arts. None of prior art discloses a method of administering the lutein and tocopherol composition for reducing the shortening rate of telomerase of cells and increasing the proliferation rate of telomerase and thereby increasing the health span of humans by way of dietary supplementation.

Furthermore, there is a need for a dietary supplement comprising Lutein as anti-oxidant and healthy-aging composition.

In summary, there is a need in the art to develop a dietary supplement that can have effect on slowing telomere shortening or antioxidant's activity to telomeric damage in turn increasing the lifespan of cells.

### SUMMARY AND OBJECTS OF THE INVENTION

The primary objective of the present invention is to provide a method of reducing, ameliorating, preventing, or reversing oxidative DNA damage in a subject, comprising orally administering an effective dose of a dietary supplement composition comprising natural lutein and tocopherols, optionally along with acceptable additives to a subject, wherein the dietary supplement composition reduces, ameliorates, prevents, or reverses the oxidative DNA damage and slows telomere shortening.

More particularly, the invention discloses the method of production of the dietary supplement composition comprising natural lutein and tocopherols, optionally along with acceptable additives, and its use in reducing telomeric damage by pro-longing the replicative lifespan of cells and slowing telomere shortening.

According to non-limiting exemplary aspect of the present invention, the natural Lutein extract is: dispersed in oil; homogenized in an aqueous medium; dispersed in an aqueous medium; processed into emulsion; encapsulated; processed into dry material; processed into solid formulation or a combination of two or more thereof.

In an embodiment, the natural Lutein extract is processed into dry material and/or solid formulation, wherein the form of the dry material is stabilized beadlets, granular powder, a powder, a cold-water dispersible powder, or a combination of two or more thereof.

In an exemplary embodiment, an oral formulation for reducing, ameliorating, preventing, or reversing oxidative DNA damage in a subject, comprising orally administering an effective dose of a dietary supplement composition comprising natural lutein and tocopherols to a subject, wherein the dietary supplement composition reduces, ameliorates, prevents, or reverses the oxidative DNA damage and slower telomere shortening.

In further embodiment, a method for promoting detoxification in cells of a subject, comprising administering to a subject an oral formulation comprising a plurality of agents that stimulate natural stem cell production within the body; repair the stem cells fibroblasts; pro-long the replicative lifespan; slow the telomere shortening; improve the telomerase activation in stem cells; improve the telomerase activation in body cells; promote the health-span in humans; and reduce the oxidative stress.

According to non-limiting exemplary aspect of the present invention, it describes use of the dietary supplement composition in stimulating natural stem cell production within the body, wherein stem cells not only combat disease, they contribute to health maintenance and reduce the effects of injury and aging.

According to embodiments of the present invention, a new approach to slowing aging and increasing longevity by increasing the number of healthy cells in the human body and also to increase the length of the telomeres in the DNA of these healthy cells. Telomeres in existing cells can be lengthened and the length of telomeres in newly produced stem cells can be increased with the daily administration of this dietary formulation. The nutritional supplements may repair telomere shortening and decrease the rate of telomere shortening.

According to yet another embodiment of the present invention, the dietary nutritional supplements disclosed below support telomere lengthening to aid in attaining a maximum life span, as well as repair telomere shortening and decrease the rate of telomere shortening.

Inventive step involved in the present invention deals with the method of administering the composition containing lutein and tocopherol crystals to subjects in the form of dietary supplement so that the oxidative stress is reduced, reduction of telomerase shortening occurs and in turn resulting in increase in the health span of humans and at the same time it can be produced on a large scale in the industry.

According to non-limiting exemplary aspect of the present invention, dietary supplement composition comprising natural lutein and tocopherols; wherein said compositions are selected from the group comprising of hard-shell capsules, softgel capsules, liquid fill capsules, tablets, effervescent granules, beverages, powdered drink mixes, melt-in-mouth sachets, juices, baked goods, gummies, jelly sticks, candies or any combinations thereof.

As would be appreciated by the person skilled in the art, the invention of Lute-gen has the following merits,

Lutein extracted by saponification from marigold flowers as it contains up to 88% of lutein esters and low quantities of other carotenoids compared to other plant sources.

Alcoholic extract of lutein makes it non-toxic and suitable dietary supplement.

Recrystallization is done from alcohol alone and not from binary solvent system as done by most other inventions.

Employed natural tocopherol as a stabilizer which is again from sunflower oil and also it acts as a natural anti-oxidant and anti-ageing agent (natural ingredient).

According to non-limiting exemplary aspect of the present invention, dietary supplement composition comprising natural lutein and tocopherols, prevents macular degeneration, skin-ageing, eye tissue protection, anti-wrinkle, free-radical scavenging, lutein extraction by various methods such as supercritical fluid CO2 extraction, changing solvent systems and others, composition deals with telomerase damage repairing from stem cells fibroblasts by providing lutein as an assimilable dietary supplement.

Several aspects of the invention are described below with reference to examples for illustration. However, one skilled in the relevant art will recognize that the invention can be practiced without one or more of the specific details or with other methods, components, materials and so forth. In other instances, well-known structures, materials, or operations are not shown in detail to avoid obscuring the features of the invention. Furthermore, the features/aspects described can be practiced in various combinations, though only some of the combinations are described herein for conciseness.

Several aspects of the invention are described below with reference to examples for illustration. However, one skilled in the relevant art will recognize that the invention can be practiced without one or more of the specific details or with other methods, components, materials and so forth. In other instances, well-known structures, materials, or operations are not shown in detail to avoid obscuring the features of the invention. Furthermore, the features/aspects described can be practiced in various combinations, though only some of the combinations are described herein for conciseness.

### BRIEF DESCRIPTION OF THE DRAWINGS

The foregoing and other objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings.

FIG. 1 Illustrates a process flow diagram illustrating one presently preferred embodiment for obtaining composition comprising natural lutein and tocopherols, optionally along with acceptable additives, according to the aspects of present invention.

FIG. 2 Depicts growth curves of human adult primary fibroblasts untreated (control-DMSO), treated with Lutein (10 μg/ml, 5 μg/ml and 1 μg/ml) under standard and oxidative (10 μM H₂O₂) condition. Each point on the population curve represents one week.

FIG. 3 Illustrates the histogram shows the distribution of telomere lengths in sample Ctrl-PO. Bars represent the relative frequency for every particular fluorescence intensity normalized (X axis). The 20th percentile (red bars) indicates the particular length below which 20% of the telomeres have been observed. The median (MTL) and average (ATL) telomere length are also indicated in the histogram that allows the analysis of telomere length variability.

FIG. 4 Shows the representative TAT image. Both the nuclei (white) and the telomeric spots (bright white spots) are seen in the image.

FIG. 5 Illustrates the bar graphs of TAT results (Median Telomere Length) for different time points and treatments.

FIG. 6 Illustrates the bar graphs of TAT results (20th Percentile Telomere Length) for different time points and treatments.

FIG. 7 Illustrates the bar graphs of TAT results (% Short Telomeres) for different time points and treatments.

FIG. 8 Illustrates the telomere shortening rate (Median of telomere length (initial-final)/Population Doubling) at the defined time points and treatments.

FIG. 9 Illustrates the graph of telomere length vs Population doubling at different treatments. Slope of regression line in each condition, represents the telomere shortening rate. Y=aX+b; a=slope and b=y intercept.

FIG. 10 shows all grades of Lute-gen, having stability data (long term and accelerated), with a minimum self-life of two years from the date of packaging.

In the drawings, like reference numbers generally indicate identical, functionally similar, and/or structurally similar elements. The drawing in which an element first appears is indicated by the leftmost digit(s) in the corresponding reference number.

### DETAILED DESCRIPTION OF THE INVENTION

It is to be understood that the present invention is not limited in its application to the details of construction and the arrangement of components set forth in the following description or illustrated in the drawings. The present invention is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced items. Further, the use of terms “first”, “second”, and “third”, and the like, herein do not denote any order, quantity, or importance, but rather are used to distinguish one element from another.

As used herein, the singular forms “a”, “an”, and “the” include both singular and plural referents unless the context clearly dictates otherwise. By way of example, “a dosage” refers to one or more than one dosage.

The terms “comprising”, “comprises” and “comprised of” as used herein are synonymous with “including”, “includes” or “containing”, “contains”, and are inclusive or open-ended and do not exclude additional, non-recited members, elements or method steps.

The term “managing” or “management” includes preventing, treating and healing of a condition or disorder or ill effects or side effects. The term also encompasses maintenance of the optimum state and prevention of the further progress in the condition or disorder or ill effects or side effects.

The term “subject” is an animal or a mammal, including human beings.

The term, “plurality of agents”, “compositional elements”, and/or materials may be presented in a common list for convenience. However, these lists should be construed as though each member of the list is individually identified as a separate and unique member. In addition, no individual member of such list should be construed as an equivalent of any other member of the same list solely based on their listing in a common group without indications to the contrary.

The term “Concentrations”, “amounts”, and other “numerical data” may be expressed or presented herein in a range format. It is to be understood that such range format is used merely for convenience and thus should be interpreted flexibly to include not only the numerical values explicitly recited as the limits of the range, but also to include all the individual numerical values or sub-ranges encompassed within that range as if each numerical value and sub-range is explicitly recited.

The term “about” means that dimensions, sizes, formulations, parameters, shapes and other quantities and characteristics are not and need not be exact, but may be approximated and/or larger or smaller, as desired, reflecting tolerances, conversion factors, rounding off, measurement error and the like and other factors known to those of skill. In addition, unless otherwise stated, the term “about” shall expressly include “exactly,” consistent with the specification regarding ranges and numerical data.

The term, “effective amount” refers to an amount of an ingredient which, when included in a composition, is sufficient to achieve an intended compositional or physiological effect. Furthermore, a “therapeutically effective amount” refers to a sufficient amount of an active agent, to achieve therapeutic results in treating or preventing or promoting a condition for which the active agent is known to be effective. It is understood that the “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments/therapy/conditions may make the choice of therapeutic effects a subjective decision. The determination of an effective amount is known within the ordinary skill in the art of pharmaceutical and nutritional sciences as well as medicine.

The term, “administration” or “administering” refer to the manner in which an active agent, or composition containing such, is presented or given to a subject.

The term, “oral administration” refers to a route of administration including swallowing, chewing, or sucking of an oral dosage form comprising the drug or nutritional formula.

The term, “stem cells’ refers to special human cells that have the ability to develop into many different cell types, for example, from muscle cells to brain cells. In some cases, they also have the innate ability to repair damaged tissues.

The term, “aflatoxins’ refers to a family of toxins produced by certain fungi that are found on agricultural crops such as maize (corn), peanuts, cottonseed, and tree nuts.

All documents cited in the present specification are hereby incorporated by reference in their totality. In particular, the teachings of all documents herein specifically referred to are incorporated by reference.

Example embodiments of the present invention are described with reference to the accompanying figures.

In the drawings, like reference numbers generally indicate identical, functionally similar, and/or structurally similar elements. The drawing in which an element first appears is indicated by the leftmost digit(s) in the corresponding reference number.

### Embodiments of the Invention

Lute-gen® is a proprietary product of company BIO-GEN EXTRACTS PRIVATE LIMITED.

In an embodiment, invention deals with a method of reducing, ameliorating, preventing, or reversing oxidative DNA damage in a subject, comprising orally administering an effective dose of a dietary supplement composition comprising natural lutein and tocopherols, optionally along with acceptable additives to a subject, wherein the dietary supplement composition reduces, ameliorates, prevents, or reverses the oxidative DNA damage and slows telomere shortening.

In an alternative embodiment, the acceptable additive is selected from group comprising gum, granulating agents, binding agents, lubricating agents, disintegrating agents, sweetening agents, coloring agents, flavoring agents, coating agents, plasticizers, preservatives, suspending agents, emulsifying agents, sweetening agents, antioxidants, surfactants, viscosity enhancers, plant cellulosic materials, additives, solvents, glidants, anti-adherents, anti-static agents, preservatives, suspending agents and spheronization agents or any combinations thereof.

In an exemplary embodiment, a method for obtaining a dietary supplement composition comprising natural lutein and tocopherols, optionally along with acceptable additives, wherein the said method comprising acts of:

a. saponifying Marigold Oleoresin in the presence of an alcohol or organic solvent and an alkali for about 2 hours at a preferable temperature of about 80 to 85 degrees centigrade;
b. adding an aqueous solution to the reaction mass, hereby obtaining a neutralized mass;
c. filtering the neutralized mass, to obtain the Lutein crystals;
d. further crystalizing the Lutein crystals in the presence of alcohol at 5 degrees centigrade to obtain a purity of 75% to 80% Lutein;
e. stabilizing the Lutein crystals of by adding natural tocopherols, and maintaining at room temperature; and
f vacuum drying the 75% to 80% purity Lutein crystals for about 2 hrs.

In an additional embodiment, the composition wherein the natural Lutein is derived from a Marigold species of Tagetes erecta and tocopherols from sunflowers, wherein the dietary supplement composition extracts are produced/separated by a process comprising supercritical carbon dioxide extraction, ethanol/methanol extraction, filtration, centrifugation, sedimentation and/or combinations thereof.

In an additional embodiment, solvents are aliphatic compounds selected from group comprising methanol, ethanol, anhydrous ethanol, propanol, butanol, ethyl acetate or combinations thereof, wherein the preferable solvent is ethanol.

In another embodiment, the dietary supplement composition extract is administered to the subject in combination with at least one additional biologically active compound, wherein the biologically active compound is a carotenoid, an antioxidant, a vitamin, mineral salts and/or trace elements, acceptable additives or a second natural extract.

In further embodiment, the natural Lutein extract is: dispersed in oil; homogenized in an aqueous medium; dispersed in an aqueous medium; processed into emulsion; encapsulated; processed into dry material; processed into solid formulation or a combination of two or more thereof.

In further embodiment, The method of any one of claims 1-7, wherein the natural Lutein extract is processed into dry material and/or solid formulation, wherein the form of the dry material is stabilized beadlets, granular powder, a powder, a cold water dispersible powder, or a combination of two or more thereof and the material has the particle size distribution in the range of 20 mesh to 100 mesh (preferably in the range of 30 mesh to 60 mesh).

In an additional embodiment, the solid formulation according to any one or more of the preceding claims, wherein the amount of total carotenoids is in the range of 5 to 85 weight-% and the dietary supplement composition is a mixture of lutein and zeaxanthin, and wherein the molar ratio of lutein to zeaxanthin is in the range of 20:1 to 1:1, preferably the molar ratio is in the range of 10:1 to 5:1.

In an additional embodiment the amount of the tocopherols is in the range of 0.1 to 10 weight-%, preferably in the range of 0.2 to 2 weight-%.

In an embodiment, an oral formulation for reducing, ameliorating, preventing, or reversing oxidative DNA damage in a subject, comprising orally administering an effective dose of a dietary supplement composition comprising natural lutein and tocopherols to the subject, wherein the dietary supplement composition reduces, ameliorates, prevents, or reverses the oxidative DNA damage and slower telomere shortening.

In an exemplary embodiment, an oral formulation wherein the formulation does not comprise any of the following compounds: hydrolyzed lecithin products, lactose-based products, animal-based products, soy-based products or marine-based products.

In a further embodiment the oral formulation is safe, non-toxic, gluten-free, solvent-free, pesticide-free, aflatoxin-free and free of adverse effects.

In an embodiment, a method for promoting detoxification in cells of a subject, comprising administering to a subject an oral formulation comprising a plurality of agents that stimulate natural stem cell production within the body; repair the stem cells fibroblasts; pro-long the replicative lifespan; slow the telomere shortening; improve the telomerase activation in stem cells; improve the telomerase activation in body cells; promote the health-span in humans; and reduce the oxidative stress.

In a further embodiment, the subject is animal or human being.

In an embodiment, wherein the effective dose of Lutein reduces the oxidative DNA damage by at least 20% compared to a subject not administered the effective dose of Lutein and the effective dose is about 6 mg to 10 mg Lutein per day.

In an exemplary embodiment, a method for promoting detoxification in cells of a subject according to claim 16, wherein the oral formulation is administered to the subject in a food or beverage product as diet plan and nutritional supplements.

In a further embodiment, a food product comprising the natural Lutein extract according to claim 20; wherein said food products & dietary supplements are selected from the group comprising of hard-shell capsules, softgel capsules, liquid fill capsules, tablets, effervescent granules, beverages, powdered drink mixes, melt-in-mouth sachets, juices, baked goods, gummies, jelly sticks, candies or any combinations thereof.

### Detailed Process of the Invention

Lute-gen® contains Lutein, a xanthophyll found in plants. Lutein is an oxygenated carotenoid found in vegetables and fruits. It is found in macula of eye where it is believed to act as a filter and is responsible for central vision. The human body cannot synthesize lutein and its needs are to be fulfilled from dietary sources.

Lute-gen® 75% (Free) Crystals were used in the in-vitro telomere length study.

Equipment Used:

Reactor

Solvent storage tanks

External jacket for heating

Centrifuge

Filtration Unit

Blending or Homogenization Unit

Vacuum Dryer

Packing in internationally accepted packing solution

Raw Materials:

Marigold Oleoresin from Marigold flowers

Tocopherols from Sunflowers

Aliphatic Alcohol

Alkali

Water

Process:

The Marigold Oleoresin is received at the warehouse and tested for Xanthophyll content & other parameters.

Method of Obtaining Lute-Gen® Crystals:

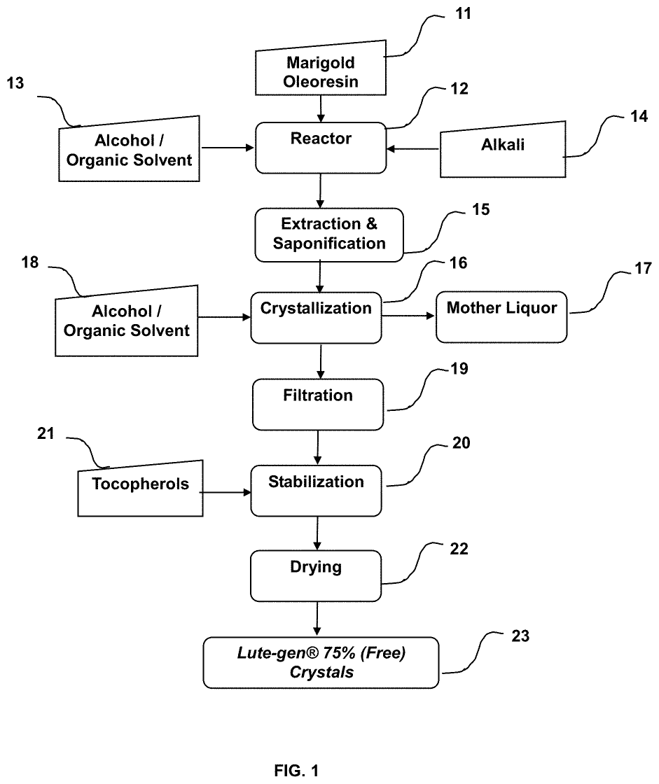

The invention discloses the method of producing a dietary supplement composition comprising natural lutein and tocopherols, (FIG. 1) comprising acts of:
a. saponifying Marigold Oleoresin in the presence of an alcohol or organic solvent and an alkali for about 2 hours at a preferable temperature of about 80 to 85 degrees centigrade; 11, 12, 13, 14, 15
b. adding an aqueous solution to the reaction mass, hereby obtaining a neutralized mass; 17, 18
c. filtering the neutralized mass, to obtain the Lutein crystals; 19
d. further crystalizing the Lutein crystals in the presence of alcohol at 5 degrees centigrade to obtain a purity of 75% to 80% Lutein; 20
e. stabilizing the Lutein crystals of by adding natural tocopherols, and maintaining at room temperature; and 20, 21
f. vacuum drying the 75% to 80% purity Lutein crystals for about 2 hrs. 22, 23

FIG. 1 shows is a process flow diagram illustrating one presently preferred embodiment for obtaining composition comprising a dietary supplement composition comprising natural lutein and tocopherols, optionally along with acceptable additives, according to the aspect of present invention.

Upon completion of the extraction, the material is unloaded to produce: Lute-gen® 75% (Free) Crystals.

Lutein acts as an antioxidant, protecting cells against damaging effects of free radicals. It is thought to function as a light filter, protecting eye tissues from sunlight damage by absorbing damaging blue, violet and ultra-violet light. It reduces the amount of blue light that reaches the photoreceptors and scavenges the reactive oxygen species, thereby preventing damage to DNA and protein molecules. It reduces risk of progression of age-related macular degeneration. In general Lutein being a good antioxidant that not helps in protecting DNA and protein molecules in the eye but also in various cell types in the body.

Nutrition and lifestyle, known to modulate aging process and age-related diseases, might also affect telomerase activity. Short and dysfunctional telomeres rather than average telomere length are associated with longevity in animal models, and their rescue by telomerase maybe sufficient to restore cell and organismal viability. Improving telomerase activation in stem cells and potentially in other cells by diet and lifestyle interventions may represent an intriguing way to promote health-span in humans.

Hence, commercial production of the purified forms of dietary carotenoids particularly lutein is of great importance. These carotenoids may be used, individually or in combination, as nutritional supplements and food colorants as well as in clinical trials where their potential health benefits in the prevention of ARMD and cancer, skin care, anti-oxidant property, diabetic retinopathy, anti-wrinkle, collagen repair, anti-ageing is gaining utmost importance.

In nature lutein abundantly occur as mono and di-esters of long chain fatty acids like palmitic, stearic, myristic, oleic, linoleic, lauric and pentadecanoic acids. The lutein and zeaxanthin which are assimilated by the body in the natural ester form are biologically hydrolysed inside the human body. Only the hydrolysed free-form of lutein and zeaxanthin are transferred to the biological sites of function. High molecular weight fatty acid groups of the esters limit the dosage of free lutein and zeaxanthin consumed. Hence, the de-esterfication process is used to hydrolyze the natural carotenoid ester forms. De-esterification of a natural carotenoid ester means a process to make free the pure carotenoid from the fatty acid backbone.

Inventors have experimental evidence with the primary objective; to determine the cellular proliferation rate and telomere length in cultures of human adult primary fibroblast cells treated with Lutein under standard and oxidative stress conditions. The results obtained from this experiment has clearly drawn conclusion that lutein has a significant protective effect on telomere length erosion in vitro under oxidative stress conditions after 8 passages.

Telomere length predicts clinical outcomes and mortality, cells with shortened telomeres can remain genetically stable if the telomere maintenance system, which includes telomerase, is fully functioning and also healthy cell function.

The present invention is further elaborated with the help of the following examples. However, these examples should not be construed to limit the scope of the present invention.

### Example 1: Effect of Dietary Supplement Composition Comprising Natural Lutein and Tocopherols, (Lute-Gen®) on Telomere Length; Proliferative and Telomere Length Analysis

Analysis of cellular proliferation rate and telomere length in cultures of human adult primary fibroblast cells treated with lutein under standard and oxidative stress conditions.

Primary cultures of adult human fibroblast were established. Cells are seeded at 2×103 cells/cm2 in GlutaMax™ high glucose Dulbecco's modified Eagle's medium (DMEM, Gibco®), and supplemented with 10% fetal bovine serum (FBS, HyClone, Thermo Scientific), penicillin (100 U/ml) and reptomycin (1000 U/ml). Media is renewed every 2-3 days and cells passaged at sub-confluence (70-80%) every 4-5 days.

Compounds or vehicle control are added to the cells in culture. Cell growth is monitored for each condition by counting cell numbers at each passage using a Countess™ cell counter (Invitrogen). Population doubling (PD) was calculated with the formula PD=(log (Nn/Nn-1))/log 2 where n is the passage; and N the number of cells. One PD is equivalent to one round of cell replication.

Materials and Methods

Treatments:

Eight independent condition were evaluated by proliferative Analysis:

Control: Standard cell culture condition (Ctrl)

Oxidative Stress Control: Standard cell culture condition+10 μM H₂O₂ (H₂O₂)

Standard cell culture condition+Lutein−10 μg/ml (LUT10)

Standard cell culture condition+Lutein−5 μg/ml (LUT5)

Standard cell culture condition+Lutein−1 μg/ml (LUT1)

Standard cell culture condition+Lutein−10 μg/ml+10 μM H₂O₂ (LUT10+H₂O₂)

Standard cell culture condition+Lutein−5 μg/ml+10 μM H₂O₂(LUT5+H₂O₂)

Standard cell culture condition+Lutein−1 μg/ml+10 μM H₂O₂ (LUT1+H₂O₂)

Cells were expanded during eight weeks under standard and oxidative (10 μM H₂O₂) cell culture condition as described previously. Fresh treatments were prepared and added to the cells at every passage and also when media was renewed. Stock solution of 10 mg/ml of the compound was prepared in DMSO and the different treatments were prepared from that stock.

Results

Tables 1, 2 and 3 show different cell passage studies.

TABLE 1
Population Doubling per passage
Population Doubling

| | | | | | | | LUT10 + | LUT5 + | LUT1 + |
|---|---|---|---|---|---|---|---|---|---|
| Week | Passage | Ctrl | H₂O₂ | LUT10 | LUT5 | LUT1 | H₂O₂ | H₂O₂ | H₂O₂ |
| 0 | P0 | 0.00 | 0.00 | 0.00 | 0.00 | 0.00 | 0.00 | 0.00 | 0.00 |
| 1 | P1 | 3.74 | 2.58 | 3.58 | 3.81 | 3.81 | 3.50 | 3.58 | 3.42 |
| 2 | P2 | 3.12 | 2.12 | 3.12 | 2.65 | 3.50 | 3.42 | 3.32 | 2.87 |
| 3 | P3 | 3.32 | 2.22 | 3.12 | 2.65 | 3.57 | 2.74 | 3.22 | 2.37 |
| 4 | P4 | 3.42 | 3.66 | 3.50 | 3.42 | 3.50 | 3.42 | 3.42 | 3.12 |
| 5 | P5 | 2.07 | 1.82 | 2.43 | 2.49 | 2.02 | 1.90 | 2.16 | 2.49 |
| 6 | P6 | 2.38 | 2.02 | 2.74 | 1.45 | 3.00 | 2.00 | 2.14 | 2.65 |
| 7 | P7 | 2.60 | 1.22 | 2.69 | 1.68 | 2.68 | 1.55 | 2.87 | 1.00 |
| 8 | P8 | 4.06 | 3.12 | 2.74 | 3.12 | 2.74 | 2.30 | 2.34 | 2.30 |

TABLE 2
Cumulative Population Doubling per passage.
Cumulative Population Doubling

| | | | | | | | LUT10 + | LUT5 + | LUT1 + |
|---|---|---|---|---|---|---|---|---|---|
| Week | Passage | Ctrl | H₂O₂ | LUT10 | LUT5 | LUT1 | H₂O₂ | H₂O₂ | H₂O₂ |
| 0 | P0 | 0.00 | 0.00 | 0.00 | 0.00 | 0.00 | 0.00 | 0.00 | 0.00 |
| 1 | P1 | 3.74 | 2.58 | 3.58 | 3.81 | 3.81 | 3.50 | 3.58 | 3.42 |
| 2 | P2 | 6.85 | 4.70 | 6.70 | 6.47 | 7.31 | 6.92 | 6.91 | 6.29 |
| 3 | P3 | 10.17 | 6.92 | 9.82 | 9.12 | 10.18 | 9.65 | 10.13 | 9.16 |
| 4 | P4 | 13.59 | 10.59 | 13.32 | 12.53 | 13.69 | 13.07 | 13.54 | 12.28 |
| 5 | P5 | 15.66 | 12.41 | 15.75 | 15.02 | 15.71 | 14.97 | 15.70 | 14.76 |
| 6 | P6 | 18.04 | 14.43 | 18.49 | 16.47 | 18.71 | 16.97 | 17.84 | 17.41 |
| 7 | P7 | 20.64 | 15.65 | 21.18 | 18.15 | 21.39 | 18.52 | 20.71 | 18.41 |
| 8 | P8 | 24.70 | 18.77 | 23.92 | 21.26 | 24.13 | 20.83 | 23.06 | 20.72 |

TABLE 3
Total number of cells per passage
Total number of cells

| | | | | | | | LUT10 + | LUT5 + | LUT1 + |
|---|---|---|---|---|---|---|---|---|---|
| Week | Passage | Ctrl | H₂O₂ | LUT10 | LUT5 | LUT1 | H₂O₂ | H₂O₂ | H₂O₂ |
| 0 | P0 | 1.50E+05 | 1.50E+05 | 1.50E+05 | 1.50E+05 | 1.50E+05 | 1.50E+05 | 1.50E+05 | 1.50E+05 |
| 1 | P1 | 2.00E+06 | 9.00E+05 | 1.80E+06 | 2.10E+06 | 2.10E+06 | 1.70E+06 | 1.80E+06 | 1.60E+06 |
| 2 | P2 | 1.73E+07 | 3.90E+06 | 1.56E+07 | 1.33E+07 | 2.38E+07 | 1.81E+07 | 1.80E+07 | 1.17E+07 |
| 3 | P3 | 1.73E+08 | 1.82E+07 | 1.35E+08 | 8.33E+07 | 1.75E+08 | 1.21E+08 | 1.68E+08 | 8.60E+07 |
| 4 | P4 | 1.85E+09 | 2.31E+08 | 1.53E+09 | 8.89E+09 | 1.98E+09 | 1.29E+09 | 1.79E+09 | 7.46E+08 |
| 5 | P5 | 7.77E+09 | 8.15E+08 | 8.27E+09 | 4.98E+09 | 8.04E+09 | 4.81E+09 | 8.00E+09 | 4.18E+09 |
| 6 | P6 | 4.04E+10 | 3.31E+09 | 5.52E+10 | 1.36E+10 | 6.44E+10 | 1.93E+09 | 3.52E+10 | 2.62E+10 |
| 7 | P7 | 2.45E+11 | 7.73E+09 | 3.57E+11 | 4.35E+10 | 4.12E+11 | 5.65E+10 | 2.58E+11 | 5.23E+10 |
| 8 | P8 | 4.08E+12 | 6.70E+10 | 2.38E+12 | 3.77E+11 | 2.75E+12 | 2.79E+11 | 1.31E+12 | 2.58E+11 |

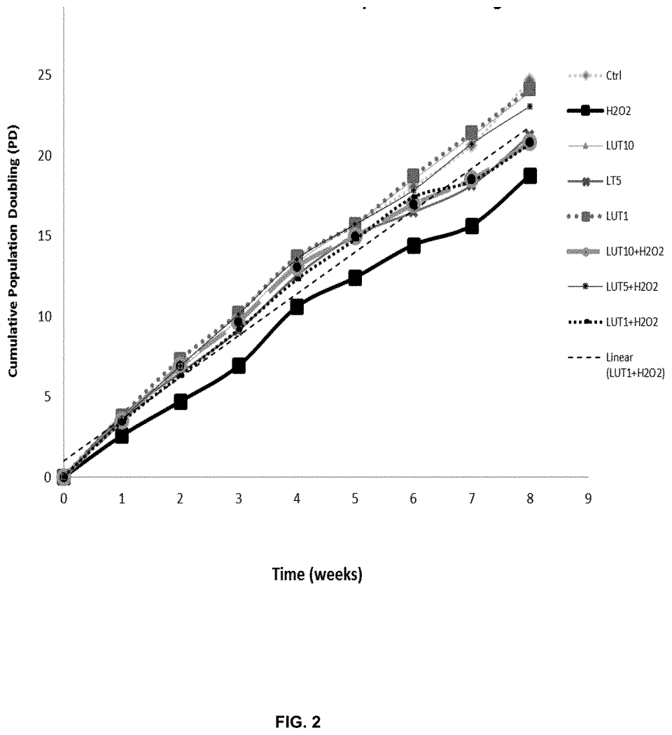

FIG. 2 shows the growth curves of human adult primary fibroblasts untreated (control-DMSO), treated. with Lutein (10 μg/ml, 5 μg/ml and 1 μg/ml) under standard and oxidative (10 μM H₂O₂) condition. Each point on the population curve represents one week.

After eight weeks of treatment with Lutein 10 μg/ml, 5 μg/ml and 1 μg/ml:

Under standard culture conditions, no difference was identified between the proliferations rates of the treated groups (LUT10, LUT5 and LUT1) compared to control (Ctrl).

Under oxidative culture conditions, all Lutein treated groups (LUT10+H2O2, LUT5+H₂O₂ and UT1+H2O2) showed an increase in proliferation rate (through oxidative protection) compared to control group (H₂O₂).

### Example 2: Effect of Dietary Supplement Composition Comprising Natural Lutein and Tocopherols, (Lute-Gen®) on Telomere Length; Telomere Length Measurements by Telomere Analysis Technology

Methods

A high-throughput (HT) Q-FISH technique was used. This method is based on a quantitative fluorescence in-situ hybridization method modified for cells in interphase2. In brief, telomeres are hybridized with a fluorescent Peptide Nucleic Acid probe (PNA) that recognizes three telomere repeats. The images of the nuclei and telomeres are captured by a high-content screen system. The intensity of the fluorescent signal from the telomeric PNA probes that hybridize to a given telomere is proportional to the length of that telomere. The intensities of fluorescence are translated to base pairs through a standard regression curve which is generated using control cell lines with known telomere length.

Sample Preparation and HT Q-FISH: On processing day, the samples and control cell lines frozen in liquid nitrogen are thawed at 37° C. and cell counts and cellular viability are determined. Aliquots with viability lower than 60% are considered below our QC standards and are not further analyzed. Cells are seeded in clear bottom black-walled 384-well plates at 15,000 cells per well in 5 replicates for each sample and 8 replicates for each control cell line.

Cells are fixed with methanol/acetic acid (3/1, vol/vol). Once these cells have fixed onto the plate, they are treated with pepsin to digest the cytoplasm and the nuclei are processed for in situ hybridization with the PNA probe. After several washing steps following standard DAPI incubation for DNA staining, the wells are filled up with mounting medium and the plate is stored overnight at 4° C.

HT Microscopy: Quantitative image acquisition and analysis is performed on a High Content Screening Opera System. Images are captured, using a 40×0.95 NA water immersion objective. UV and 488 nm excitation wavelengths are used to detect the DAPI and A488 signals respectively. With constant exposure settings, 15 independent images are captured at different positions for each well. Next, the nuclei images are used to define the region of interest for each cell, measuring telomere fluorescence intensity of the A488 image in all of them. The results of intensity for each foci are exported to the Columbus 2.4 software. Telomere length distribution and median telomere length are calculated with Life Length's proprietary program.

Validation

The TAT technology has been validated for the following parameters:

Accuracy: The establishment of a correspondence between TAT fluorescence intensity values and telomere length measurements is achieved by performing TRF (Terminal Restriction Fragmentation) in six human lymphocyte cell lines (Calibration/Method Comparison). The same set of samples is analyzed both by TAT and by the TRF reference method (Definition of TAT Systemic Error).

VALIDATION DATA shows a correlation of 0.99.

Precision: Serial analysis of the median telomere length values is performed on a human lymphocyte sample in different runs, days and plate positions in order to define TAT Random Error parameters (Standard Deviation, Variance).

VALIDATION DATA indicates that TAT has a standard deviation of 454 base pairs. Limit of Detection and Specificity: Definition of image analysis algorithms and protocol settings exist that define the lowest significant spot intensities and avoid interference by nonspecific fluorescence signals.

VALIDATION DATA defines the limit of detection at 800 base pairs and demonstrates very high specificity.

Median Reportable Range: Analysis of median telomere length of 6 cell lines is performed that covers our reportable range and defines its lower and upper limits.

VALIDATION DATA fix lower level at 4,700 base pairs and upper level at 14,400 base pairs.

Reference Range: Analysis of median telomere length have been conducted in hundreds of human samples in order to define the TAT Reference Range and its percentiles (5th, 10th, 25th, 50th 75th and 95th) for different ages.

VALIDATION DATA established population curves—normal population data base from 18 to 85 years, to extrapolate patients' data and generate reports.

Results

During the TAT protocol, the samples were assessed for:

Cell count: An automated cell counter is used to determine the total number of cells in the vials. A minimum of 100.000 cells are needed to perform TAT.

Cell viability by Tripan-Blue exclusion method. The exclusion criteria currently in place will reject aliquots with less than 60% viable cells.

Regression Curve: Internal controls are included and a regression analysis is performed for each run/plate. The plates are repeated if their regression curves have an R2 below 0.92.

Replicates: After seeding and once TAT is completed:

Samples with CV above 10% are discarded.

Samples with less than 3 valid replicates at the end of the analysis are discarded. Spot number analyzed per sample should be higher than 1,000.

Cell Count and Viability Results

All samples were analyzed by TAT.

TABLE 4
Concentration and viability of samples after thawing

| | | Cell conc | Viability |
|---|---|---|---|
| LL CODE | Week | (cells/mL) | % |
| Ctrl-P0 | Control - Week 0 | 1.4 | 91 |
| Ctrl-P4 | Control - Week 4 | 0.66 | 91 |
| H₂O₂-P4 | 10 μm H₂O₂ - Week 4 | 0.92 | 92 |
| LUT10-P4 | LUT (10 μg/ml) - Week 4 | 0.95 | 96 |
| LUT5-P4 | LUT (5 μg/ml) - Week 4 | 0.98 | 96 |
| LUT1-P4 | LUT (1 μg/ml) - Week 4 | 0.24 | 80 |
| LUT10 + H₂O₂-P4 | LUT (10 μg/ml) + 10 μMH₂O₂ - | 0.21 | 96 |
| | Week 4 | | |
| LUT5 + H₂O₂-P4 | LUT (5 μg/ml) + 10 μMH₂O₂ - | 0.22 | 81 |
| | Week 4 | | |
| LUT1 + H₂O₂-P4 | LUT (1 μg/ml) + 10 μMH₂O₂ - | 0.31 | 87 |
| | Week 4 | | |
| Ctrl + P8 | Control - Week 8 | 0.25 | 83 |
| H₂O₂-P8 | 10 μM H₂O₂ - Week 8 | 0.57 | 87 |
| LUT10-P8 | LUT (10 μg/ml) - Week 8 | 0.53 | 87 |
| LUT5-P8 | LUT (5 μg/ml) - Week 8 | 0.73 | 91 |
| LUT1-P8 | LUT (1 μg/ml) - Week 8 | 1.2 | 91 |
| LUT10 + H₂O₂-P8 | LUT (10 μg/ml) + 10 μMH2O2 - | 0.67 | 92 |
| | Week 8 | | |
| LUT5 + H₂O₂-P8 | LUT (5 μg/ml) + 10 μMH2O2 - | 0.28 | 76 |
| | Week 8 | | |
| LUT1 + H₂O₂-P8 | LUT (1 μg/ml) + 10 μMH2O2 - | 0.53 | 87 |
| | Week 8 | | |

TABLE 5
Plate's Quality Control Parameters
1804048-PTAT

| | |
|---|---|
| A Coef | 6.50 |
| B Coef | 7.62 |
| R2 | 0.99 |
| Control median | 414 |
| Sample median | 327 |

Telomere Length Results from TAT Analysis

The following table (Table 6) displays the TAT parameters from the Histogram. Median telomere length, 20^{th} percentile telomere length, percentage of short telomeres and the coefficients of variation for each set of samples. The percentage of short telomeres is defined as the percentage of those below 3 Kbp (<3 Kbp). As mentioned earlier, measurements in five replicates per sample were performed.

TABLE 6
Telomere length results from TAT analysis. All samples meet the quality control parameters

| | | Median Length | 20th Percentile | Telomeres | CV |
|---|---|---|---|---|---|
| LL-CODE | Treatment | (base pairs) | Length (base pairs) | <3 Kbp (%) | (%) |
| Ctrl-P0 | Control - Week 0 | 7338 | 3789 | 14.37 | 0.40 |
| Ctrl-P4 | Control - Week 4 | 6519 | 3381 | 15.83 | 1.37 |
| H₂O₂-P4 | 10 μM H₂O₂ - Week 4 | 6369 | 3167 | 18.62 | 1.61 |
| LUT10-P4 | LUT (10 μg/ml) - Week 4 | 6330 | 3198 | 18.48 | 0.15 |
| LUT5-P4 | LUT (5 μg/ml) - Week 4 | 6349 | 3104 | 18.98 | 0.85 |
| LUT1-P4 | LUT (1 μg/ml) - Week 4 | 5977 | 3351 | 16.87 | 1.95 |
| LUT10 + H2O2-P4 | LUT (10 μg/ml) + 10 μMH₂O₂ - Week 4 | 5772 | 3073 | 19.10 | 5.20 |
| LUT5 + H2O2-P4 | LUT (5 μg/ml) + 10 μMH₂O₂ - Week 4 | 6077 | 3351 | 16.97 | 4.17 |
| LUT1 + H2O2-P4 | LUT (1 μg/ml) + 10 μMH₂O₂ - Week 4 | 5997 | 3381 | 16.72 | 4.70 |
| Ctrl + P8 | Control - Week 8 | 6330 | 3259 | 17.87 | 1.62 |
| H₂O₂-P8 | 10 μM H₂O₂ - Week 8 | 5429 | 2782 | 21.93 | 0.40 |
| LUT10-P8 | LUT (10 μg/ml) - Week 8 | 5997 | 2977 | 20.21 | 2.58 |
| LUT5-P8 | LUT (5 μg/ml) - Week 8 | 6037 | 2945 | 20.51 | 1.64 |
| LUT1-P8 | LUT (1 μg/ml) - Week 8 | 6077 | 2848 | 21.29 | 1.00 |
| LUT10 + H2O2-P8 | LUT (10 μg/ml) + 10 μMH2O2 - Week 8 | 5977 | 2977 | 20.09 | 2.12 |
| LUT5 + H2O2-P8 | LUT (5 μg/ml) + 10 μMH2O2 - Week 8 | 5977 | 2977 | 20.09 | 2.13 |
| LUT1 + H2O2-P8 | LUT (1 μg/ml) + 10 μMH2O2 - Week 8 | 6037 | 2848 | 21.14 | 0.83 |

### Example 3: Effect of Dietary Supplement Composition Comprising Natural Lutein and Tocopherols, (Lute-Gen®) on Telomere Length; Analysis of Telomere Length Variability

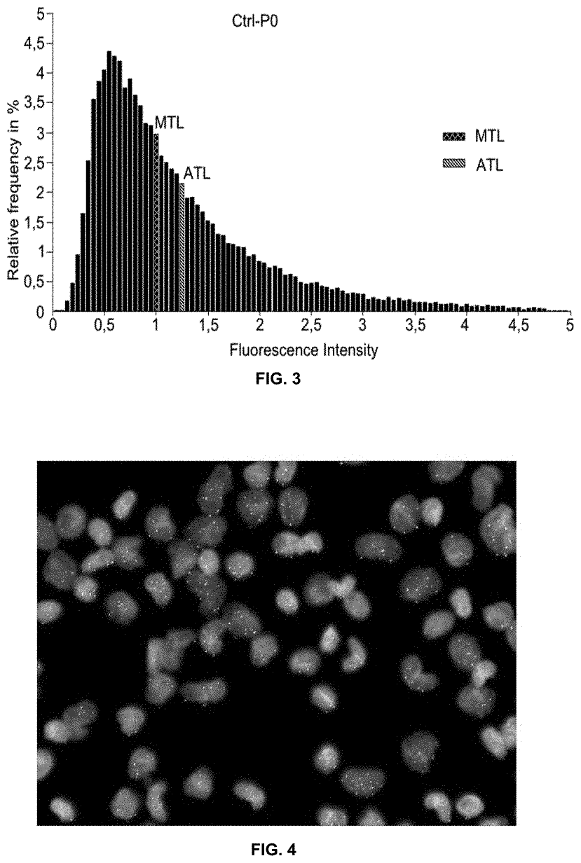

FIG. 3 Histogram shows the distribution of telomere lengths in sample Ctrl-PO. Bars represent the relative frequency for every particular fluorescence intensity normalized (X axis).

The 20th percentile (red bars) indicates the particular length below which 20% of the telomeres have been observed. The median (MTL) and average (ATL) telomere length are also indicated in the histogram. This histogram also allows for the analysis of telomere length variability. FIG. 4 shows representative TAT image, both the nuclei (white) and the telomeric spots (Bright white spots) can be seen in the image.

Data Analysis

For TAT analysis, data were grouped by condition (Ctrl, H2O2, LUT10, LUT5, LUT1, LUT10+H2O2, LUT5+H2O2, LUT1+H2O2) and time point of treatment (0, 4, and 8 weeks).

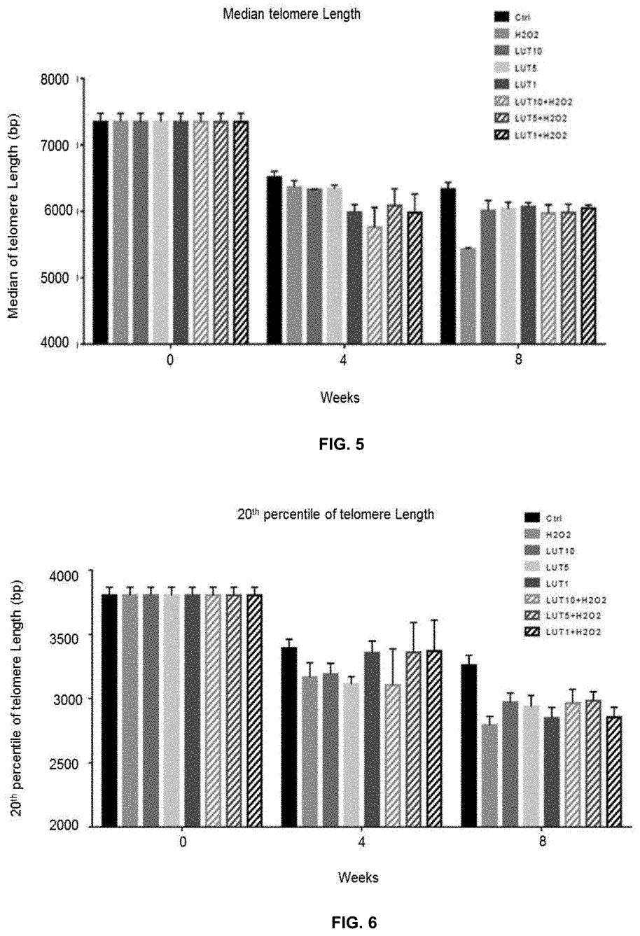

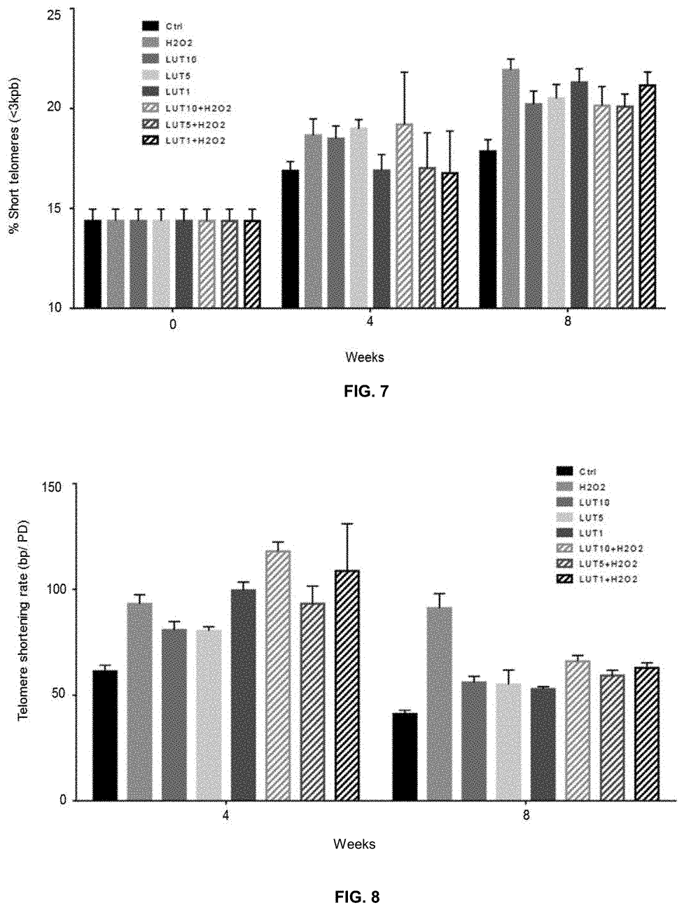

Bar graphs of TAT results (Median Telomere Length, 20th Percentile Telomere Length and % Short Telomeres) for different time points and treatments (FIGS. 5, 6 and 7).

Two-Way ANOVA Analysis was used to compare the results obtained from the TAT assay. This analysis indicates if there are significant differences among the different treatments. Significant differences are indicated in the Significance and Adj. P Value columns. From lowest to highest significance; ns: non-significant; *: p<0.05; **: p<0.01; ***p<0.001; ****p<0.0001.

Due to the fact that DNA replication during the cell cycle causes of telomere shortening, the telomere length measurements performed were normalized by the total number of population doubling (cell replication) in each condition and week.

Table 7 Summary table of cumulative population doubling and median of telomere length at different week (Week 0, 4 and 8) and treatments (Ctrl, H2O2, LUT10, LUT5, LUT1, LUT10+H2O2, LUT5+H2O2, LUT1+H2O2).

Cumulative Population Doubling

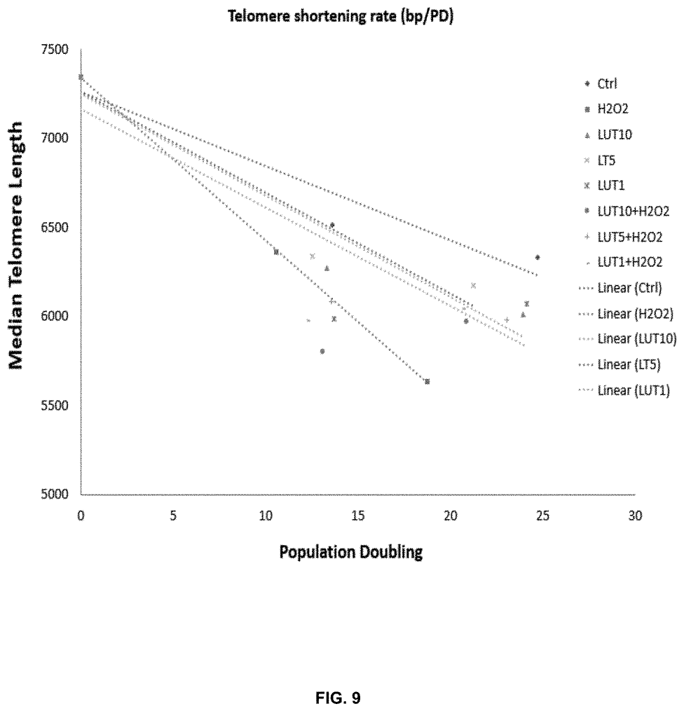

Telomere shortening rate was analysed, Median of telomere length (initial-final)/Population Doubling, at the defined time points and treatments, FIG. 8.

TABLE 7A
Telomere shortening rate (Median of telomere
length (initial-final)/Population Doubling)

| | | | | | | | | |
|---|---|---|---|---|---|---|---|---|
| | | | | | | LUT10 + | LUT5 + | LUT1 + |
| Week | Ctrl | H₂O₂ | LUT10 | LUT5 | LUT1 | H₂O₂ | H₂O₂ | H₂O₂ |

Cumulative Population Doubling

| | | | | | | | | |
|---|---|---|---|---|---|---|---|---|
| 0 | 0 | 0 | 0 | 0 | 0 | 0 | 0 | 0 |
| 4 | 13.6 | 10.6 | 13.3 | 12.5 | 13.7 | 13.1 | 13.5 | 12.3 |
| 8 | 24.7 | 18.8 | 23.9 | 21.3 | 24.1 | 20.8 | 23.1 | 20.7 |

Median Telomere Length

| | | | | | | | | |
|---|---|---|---|---|---|---|---|---|
| 0 | 7347 | 7347 | 7347 | 7347 | 7347 | 7347 | 7347 | 7347 |
| 4 | 6515 | 6360 | 6272 | 6340 | 5984 | 5804 | 6085 | 5980 |
| 8 | 6333 | 5636 | 6008 | 6177 | 6071 | 5972 | 5980 | 6045 |

Telomere Shortening Rate (bp/PD)

Telomere shortening rate (bp/PD) is shown in Table 7B.

TABLE 7B
Telomere shortening rate (bp/PD)

| | | | | | | | LUT10 + | LUT5 + | LUT1 + |
|---|---|---|---|---|---|---|---|---|---|
| Week | Passage | Ctrl | H₂O₂ | LUT10 | LUT5 | LUT1 | H₂O₂ | H₂O₂ | H₂O₂ |
| 0 | P0 | 0 | 0 | 0 | 0 | 0 | 0 | 0 | 0 |
| 4 | P4 | 61 | 93 | 81 | 80 | 100 | 118 | 93 | 111 |
| 8 | P8 | 41 | 91 | 56 | 55 | 53 | 66 | 59 | 63 |

### Example 4: Effect of Dietary Supplement Composition Comprising Natural Lutein and Tocopherols, (Lute-Gen®) on Telomere Length; Telomere Shortening Rate at Different Treatments

Graph of telomere length vs Population doubling at different treatments. Slope of regression line in each condition, represents the telomere shortening rate. Y=aX+b; a=slope andbb=y intercept, FIG. 9.

TABLE 8
Telomere shortening rate at different treatments.

| Condition | Slope (Telomere Shortening Rate) | R² |
|---|---|---|
| Ctrl | y = −41.782x + 7264.9 | R² = 0.914 |
| H₂O₂ | y = −91.272x + 7340.7 | R² = 0.999 |
| LUT10 | y = −57.039x + 7250 | R² = 0.928 |
| LUT5 | y = −56.796x + 7261.1 | R² = 0.917 |
| LUT1 | y = −55.261x + 7163.8 | R² = 0.768 |
| LUT10 + H2O2 | y = −71.479x + 7181.9 | R² = 0.999 |
| LUT5 + H2O2 | y = −61.587x + 7222 | R² = 0.879 |
| LUT1 + H2O2 | y = −66.365x + 7187.3 | R² = 0.804 |

Observations and Conclusions

The results obtained during the initial analysis of the samples indicated cell viabilities within acceptable range for all samples. The standard regression curve's coefficient of determination (R2) for the plate analyzed, met the quality control standards (R2>0.98).

Summarized Version of the Results:

Telomere analysis following 8 weeks of treatments:

Oxidative Stress decreased proliferation capacity and increased telomere shortening rate in cultures of human primary fibroblast.

For control in standard culture condition (Crtl):

The median telomere length decreased by 1014 bp (ie: from 7347 bp to 6333 bp).

The 20th percentile median telomere length shortened by 530 bp (ie: from 3789 bp to 3259 bp).

The percentage of short telomeres (<3 kpb) increased by 4% (ie: from 14% to 18%).

For control in oxidative culture condition (H₂O₂):

The median telomere length decreased by 1712 bp (ie: from 7347 bp to 5636 bp).

The 20th percentile median telomere length shortened by 1007 bp (ie: from 3789 bp to 2782 bp).

The percentage of short telomeres (<3 kpb) increased by 7% (ie: from 14% to 21%).

For treated groups in standard conditions (LUT10, LUT5, LUT1):

A significant increase in telomere shortening rate was observed after LUT1 treatment.

No other significant differences were observed under these conditions

For treated groups in oxidative culture condition (LUT10+H₂O₂, LUT5+H₂O₂, LUT1+H₂O₂):

Oxidative Stress decreased proliferation capacity and increased telomere shortening rate.

A significant difference in the telomere shortening rate was identified between LUT10+H₂O₂, LUT5+H₂O₂ and LUT1+H₂O₂ compared to control (H₂O₂) after 8 passages.

According to the data obtained from this assay, Lutein has a significant protective effect on telomere length erosion in vitro under oxidative stress conditions after 8 passages.

According to non-limiting exemplary aspect of the present invention, it describes a method of increasing telomere length in humans comprising administering an effective dietary composition comprising Lutein crystal.

According to non-limiting exemplary aspect of the present invention, it describes a particularly effective dietary supplement in stimulating natural stem cell production within the body.

According to embodiments of the present invention, a new approach to slowing aging and increasing longevity by increasing the number of healthy cells in the human body and also to increase the length of the telomeres in the DNA of these healthy cells. Telomeres in existing cells can be lengthened and the length of telomeres in newly produced stem cells can be increased with the daily administration of this dietary formulation. The nutritional supplements may repair telomere shortening and decrease the rate of telomere shortening.

According to yet another embodiment of the present invention, the dietary nutritional supplements disclosed below support telomere lengthening to aid in attaining a maximum life span, as well as repair telomere shortening and decrease the rate of telomere shortening.

According to a non-limiting exemplary aspect of the present invention describes a novel Lute-gen® dietary supplement for its antioxidant's activity to telomeric damage, pro-longing the replicative lifespan and slowing telomere shortening. Improving telomerase activation in stem cells and potentially in other cells by diet and lifestyle interventions may represent an intriguing way to promote health-span in humans.

According to further non-limiting exemplary aspect of the present invention, dietary supplement for its antioxidant's activity to telomeric damage.

Inventive step involved in the present invention deals with the method of administering the composition containing lutein and tocopherol crystals to subjects in the form of dietary supplement so that the oxidative stress is reduced, reduction of telomerase shortening occurs and in turn resulting in increase in the health span of humans.

The invention of Lute-gen® has enormous potential as the crystals of lutein+tocopherol can be used as dietary supplement to increase the health span of humans and at the same time it can be produced on a large scale in the industry.

In an embodiment, all grades of Lute-gen®, having stability data (long term and accelerated), with a minimum shelf-life of two years from the date of packaging.

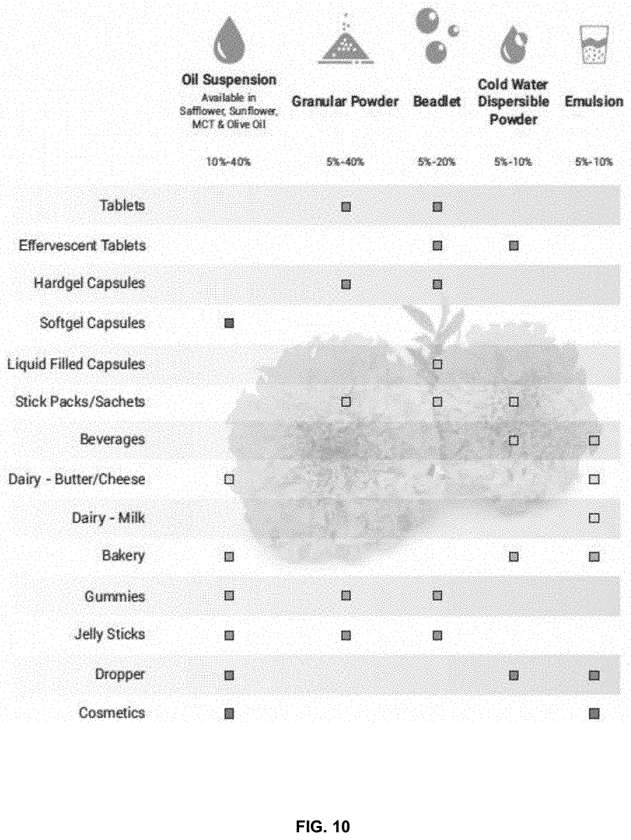

Tablets, effervescent tablets, hard-shell capsules, softgel capsules, liquid filled capsules, stick packs and sachets, beverages, dairy (butter and cheese), bakery, gummies, jelly sticks, droppers and cosmetics (FIG. 10).

According to a non-limiting exemplary aspect of the present invention can be used for the development of diet plan and nutritional supplements.

Merely for illustration, only representative number/type of graph, chart, block, and sub-block diagrams were shown. Many environments often contain many more block and sub-block diagrams or systems and sub-systems, both in number and type, depending on the purpose for which the environment is designed.

While specific embodiments of the invention have been shown and described in detail to illustrate the inventive principles, it will be understood that the invention may be embodied otherwise without departing from such principles.

Reference throughout this specification to “one embodiment”, “an embodiment”, or similar language means that a particular feature, structure, or characteristic described in connection with the embodiment is included in at least one embodiment of the present invention. Thus, appearances of the phrases “in one embodiment”, “in an embodiment” and similar language throughout this specification may, but do not necessarily, all refer to the same embodiment.

It should be understood that the figures and/or screen shots illustrated in the attachments highlighting the functionality and advantages of the present invention are presented for example purposes only. The present invention is sufficiently flexible and configurable, such that it may be utilized in ways other than that shown in the accompanying figures.

It should be understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### REFERENCES

1) Estrada J C, Albo C, Benguria A, Dopazo A, Lopez-Romero P, Carrera-Quintanar L, Roche E, Clemente E P, Enriquez J A, Bernad A & Smaper E. (2012). Culture of human mesenchymal stem cells at low oxygen tension improves growth and genetic stability by activating glycolysis. Cell Death and Differentiation. December 02; 19, 743-755.
2) Canela A, Vera E, Klatt P, Blasco M A. (2007). High-throughput telomere length quantification by FISH and its application to human population studies. Proc Natl Acad Sci USA. March 27; 104(13):5300-5.
3) Kimura M1, Stone R C, Hunt S C, Skurnick J, Lu X, Cao X, Harley C B, Aviv A. (2010). Measurement of telomere length by the Southern blot analysis of terminal restriction fragment lengths. Nature Protocols 5(9):1596-607

I/We claim:

1) A method of reducing, ameliorating, preventing, or reversing oxidative DNA damage in a subject, comprising orally administering an effective dose of a dietary supplement composition comprising natural lutein and tocopherols, optionally along with acceptable additives to a subject, wherein the dietary supplement composition reduces, ameliorates, prevents, or reverses the oxidative DNA damage and slows telomere shortening.

2) The method of claim 1, wherein the dietary supplement composition comprising natural lutein is produced by a method comprising:
a. saponifying Marigold Oleoresin in the presence of an alcohol or organic solvent and an alkali for about 2 hours at a preferable temperature of about 80 to 85 degrees centigrade;
b. adding an aqueous solution to the reaction mass, hereby obtaining a neutralized mass;
c. filtering the neutralized mass, to obtain the Lutein crystals;
d. further crystalizing the Lutein crystals in the presence of alcohol at 5 degrees centigrade to obtain a purity of 75% to 80% Lutein;
e. stabilizing the Lutein crystals of by adding natural tocopherols, and maintaining at room temperature; and
f. vacuum drying the 75% to 80% purity Lutein crystals for about 2 hrs.

3) The method according to claim 1, wherein the natural Lutein is derived from a Marigold species of Tagetes erecta.

4) The method according to claim 1, wherein the dietary supplement composition extracts are produced/separated by a process comprising supercritical carbon dioxide extraction, ethanol/methanol extraction, filtration, centrifugation, sedimentation and/or combinations thereof.

5) The method according to claim 1, wherein the dietary supplement composition extract is administered to the subject in combination with at least one additional biologically active compound.

6) The method according to claim 5, wherein the biologically active compound is a carotenoid, an antioxidant, a vitamin, mineral salts and/or trace elements, acceptable additives or a second natural extract.

7) The method of claim 1, wherein the natural Lutein is: dispersed in oil; homogenized in an aqueous medium; dispersed in an aqueous medium; processed into emulsion; encapsulated; processed into dry material; processed into solid formulation or a combination of two or more thereof.

8) The method of claim 7 wherein the form of the dry material is stabilized beadlets, granular powder, a powder, a cold water dispersible powder, or a combination of two or more thereof.

9) The method according to claim 7, wherein the granules have a particle size distribution in the range of 20 mesh to 100 mesh (preferably in the range of 30 mesh to 60 mesh).

10) The method according to claim 6, wherein the amount of total carotenoids is in the range of 5 to 85 weight-%.

11) The method according to claim 1, wherein the dietary supplement composition is a mixture of lutein and zeaxanthin, and wherein the molar ratio of lutein to zeaxanthin is in the range of 20:1 to 1:1, preferably the molar ratio is in the range of 10:1 to 5:1.

12) The method according to claim 1, wherein the amount of the tocopherols is in the range of 0.1 to 10 weight-%, preferably in the range of 0.2 to 2 weight-%.

13) The method of claim 1, comprising orally administering an effective dose of the dietary supplement composition comprising natural lutein and tocopherols to a subject.

14) The method according to claim 1, wherein the composition does not comprise any of the following compounds: hydrolyzed lecithin products, lactose-based products, animal-based products, soy-based products or marine-based products.

15) The method according to claim 1, wherein the formulation is safe, non-toxic, gluten-free, solvent-free, pesticide-free, aflatoxin-free and free of adverse effects.

16) (canceled)

17) The method according to claim 1, wherein the subject is animal or human being.

18) The method according to claim 1, wherein the effective dose of Lutein reduces the oxidative DNA damage by at least 20% compared to a subject not administered the effective dose of Lutein.

19) The method according to claim 1, wherein the effective dose is about 6 mg to 10 mg Lutein per day.

20) The method according to claim 1, wherein the oral administration to the subject in a food or beverage product as diet plan and nutritional supplements.

21) The method according to claim 20; wherein said food products & dietary supplements are selected from the group comprising of hard-shell capsules, softgel capsules, liquid fill capsules, tablets, effervescent granules, beverages, powdered drink mixes, melt-in-mouth sachets, juices, baked goods, gummies, jelly sticks, candies or any combinations thereof.
